(19) United States
(12) Patent Application Publication
(10) Pub. No.: US 2023/0103671 A1
(43) Pub. Date: Apr. 6, 2023

(54) METHODS FOR QUANTIFYING THE IMPACT OF SHEAR STRESS ON MAMMALIAN CELL LINES

(71) Applicant: Regeneron Pharmaceuticals, Inc., Tarrytown, NY (US)

(72) Inventors: Ian O'Shea, Dooradoyle Limerick (IE); John Crowley, Limerick (IE); Martin Power, Limerick (IE); Alan Ronan, Limerick (IE)

(21) Appl. No.: 17/957,490

(22) Filed: Sep. 30, 2022

Related U.S. Application Data
(60) Provisional application No. 63/251,169, filed on Oct. 1, 2021.

(51) Int. Cl. C12M 1/42; C12M 1/34

(52) U.S. Cl. CPC C12M 35/04; C12M 41/40

(57) ABSTRACT

Methods for characterizing mechanical properties of cells at different stress levels. The disclosed inventions can determine the impact of shear stress on cells in bioproduction processes.

### CROSS-REFERENCE TO RELATED APPLICATIONS

This application claims benefit of and priority to U.S. Provisional Patent Application No. 63/251,169 filed on Oct. 1, 2021, which is incorporated by reference in its entirety.

### TECHNICAL FIELD OF THE INVENTIONS

This inventions are generally related to systems and methods of characterizing the effects of shear stress on cells.

### BACKGROUND OF THE INVENTIONS

Hydrodynamic forces generated by bioproduction processes and cell culture techniques can have detrimental consequences on cellular integrity, recombinant protein production and overall viability of a cell line. Thus, there exists a need in the art to quantify the effects of hydrodynamic forces, for example, shear stress, on cell lines.

Therefore, it is an object of the inventions to provide methods for identifying the susceptibility of cells to varying rates of shear stress. The inventions further contemplate utilizing the results from quantifying shear stress to inform and improve the bioproduction process.

### SUMMARY OF THE INVENTIONS

The present inventions provide methods of quantifying the impact of shear stress on cells. In embodiments disclosed herein, the inventions comprise the steps of exposing immobilized cells to forces that cause shear stress and nanoindenting the immobilized cells to determine their mechanical properties at different stress levels.

In several embodiments disclosed herein, the cells are mammalian cells. In certain embodiments, the mammalian cells are Chinese Hamster Ovary (CHO) cells, Baby hamster kidney (BHK) cells, Human embryonic kidney 293 (HEK293) cells, HeLa cells, per.c6 cells, nonsecreting murine myeloma (NSo) cells, or Sp2/0 murine myeloma cells. In further embodiments, the cells are suspension cells.

In further embodiments, the cells are suspension cells. In yet another embodiment, the cells are immobilized using a cell and tissue adhesive (CTA). In several embodiments, the cell and tissue adhesive is Cell-Tak. In several embodiments disclosed herein, the cells are CHO cells.

In several embodiments disclosed herein, the forces that cause shear stress to cells are generated by shake flask agitation. In another embodiment, forces that cause shear stress to cells are generated by a fluidic pump system. In yet another embodiment, forces that cause shear stress to cells are generated by bioreactor agitation.

In several embodiments disclosed herein, nanoindenting the cells is performed by a nanoindenter. In some embodiments, the nanoindenter comprises an optical probe. In several embodiments, the optical probe comprises a cantilever. In one embodiment, the probe is mechanically lowered from a pre-calibrated distance toward the surface of the cells. In similar embodiments, the probe is mechanically lowered for a period of about two seconds.

In several embodiments disclosed herein, the cell exerts a force upon the cantilever upon contact with the cantilever, causing the cantilever to bend. In similar embodiments, the cantilever is in contact with the cell for about one second to about five seconds.

In some embodiments disclosed herein, the cantilever is in contact with the cell for about six seconds. In similar embodiments, the cantilever generates increasing oscillation frequencies of about 1F Hz, about 2F Hz, about 4F Hz, and about 10F Hz. In a further embodiment, no oscillation frequency is generated for a period of about two seconds between each increasing oscillation frequency.

In yet another embodiment of the methods disclosed herein, the nanoindenter subjects the cells to about six rounds of nanoindentation. In similar embodiments, each subsequent nanoindendation is placed about 2 μm from the preceding nanoindentation.

In several embodiments disclosed herein, the mechanical properties of the cells are determined after several rounds of nanoindentation. In some embodiments, the mechanical properties of the cells comprise cell stiffness. In further embodiments, cell stiffness is measured by Young's modulus (YM) and Effective Young's modulus (EYM). In some embodiments, the YM and EYM of cells after about 26 hours of shear stress is less than about 50×Pa. In other embodiments, the YM and EYM of cells after about 46 hours of shear stress is less than about 50×Pa. In another embodiment, the YM and EYM of cells after 72 hours of shear stress is greater than about 500×Pa.

In several embodiments disclosed herein, cell stiffness is determined by calculating storage modulus (E′). In some embodiments, cell stiffness is determined by calculating loss modulus (E″). In certain embodiments, the E′ value is higher than the E″ value at frequencies of about 1F, about 2F, and about 10F Hz after at least about two days of agitation, indicating elasticity of the cells. In other certain embodiments, the E″ value is higher than the E′ value at a frequency of about 4F Hz after at least about two days of agitation, indicating viscosity of the cells.

The present disclosure additionally provides methods of producing a line of adherent cells, wherein the methods comprise the steps of (a) seeding suspension cells in flasks at a seeding density of about 2D×10⁵ cells/mL to about 6D×10⁵ cells/mL; (b) introducing to the flasks a chemically-defined culture medium supplemented with a concentration of about 0.5Y% fetal bovine serum (FBS) to about 4Y% FBS; (c) measuring the viable cell density (VCD) of suspension cells using a bioanalyzer; (d) allowing the cells to grow to an adherent/suspension-cell confluency of no more than 85% total confluency; (e) passaging the culture media to remove suspension cells from the flask; (f) submerging the flask in phosphate buffer saline (PBS); (g) measuring the VCD of remaining adherent cells; and (h) repeating steps b-g for at least 72 hours and for up to six passages.

In some aspects of the inventions, the density of adherent cells is at least 13.84D×10⁵ cells/mL after six passages.

Other aspects of the inventions disclosed herein describe cells produced by the methods disclosed herein.

The present disclosure additionally provides processes of bioproduction optimization comprising inflicting shear stress on cells, quantifying the impact of shear stress on cells, and using shear stress data to adjust the levels of shear force applied during bioproduction.

In some embodiments, optimization results in an increase in product titer and yield. In another embodiment, optimization results in an increase in cell viability. In yet another embodiment, optimization results in an increase in product quality. In further embodiments, product quality is determined by glycosylation efficiency.

The present disclosure further provides methods of developing cell lines that are resistant to shear stress comprising inflicting shear stress on said cells to increasing levels of shear force, quantifying the impact of shear stress on cells, and selecting resistant cells for further use in bioproduction.

### BRIEF DESCRIPTION OF THE DRAWINGS

FIG. 1A shows, on the left, a microscopic view of a contaminated Cell Line B sample, which was seeded at 4D×10⁵ cells/mL on a glass slide inside the dish, and on the right, a microscopic view of a Cell Line A sample which was seeded at 4D×10⁵ cells/mL on a glass slide inside the dish. FIG. 1B shows, on the left, a microscopic view of the nanoindenter probe, showing the cantilever spike, and on the right, a microscopic view of suspended cells of Cell Line A (previously adapted to adherent) latching onto the probe during a nanoindentation attempt, particularly surrounding the cantilever.

FIG. 2 shows a top-down diagrammatic and microscopic view of cells seeded onto the CTA coating.

FIG. 3A is an overview of the Shake Flask sample-extraction and preparation for nanoindentation. FIG. 3B shows displacement-versus-time graphs obtained from standard indentations over a six-second period on 3 daily samples. FIG. 3C shows a focus on the Day 1 displacement-versus-time graph, highlighting the initial indentation recording. FIG. 3D shows a comparison of the simplified diagrammatic load-indentation curve, and the curve obtained from the Day 1 standard indentation.

FIG. 4A shows displacement-versus-time data retrieved from indentations on target cells, displaying the increased frequency (Hz) over time on a single point of a cell surface. FIG. 4B shows the Young's modulus versus Tan delta of Storage and Loss Modulus data retrieved by the software from the MIOF experiment.

FIG. 5 is a bar chart showing the mean Young's Modulus (YM) and Effective Young's Modulus (EYM) documented from a programmed series of six indentations on a single target Cell Line A cell sampled over three days.

FIG. 6 is an outline of the initial adherent Cell Line A experimental conditions.

FIG. 7 is a display of the growth pattern of Cell Line A cells over the first 72 hours, seeded at three different initial seeding densities in different concentrations of FBS.

FIG. 8A is a diagrammatic overview of the vial thaw directly into T75 flasks, in FBS concentrations of 1Y%, 2Y%, 3Y% and 4Y%, with the absence of FBS (0% FBS) from media acting as a control. FIG. 8B is a graph displaying the viable cell density of suspension and adherent cells present across seven passages at separate FBS concentrations.

### DETAILED DESCRIPTION OF THE INVENTIONS

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present inventions. All publications mentioned are incorporated herein by reference in their entirety.

The use of the terms “a,” “an,” “the,” and similar referents in the context of describing the presently claimed inventions (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

The use of the letter “Y” in the context of referring to the percentage of fetal bovine serum (FBS) in culture media represents the number “3” as a multiplier such that, for example, 1.5Y% FBS means 4.5% FBS.

The use of the letter “D” in the context of referring to cell density in cell culture represents the number “0.25” as a multiplier such that, for example, 4D×10⁵ cells/mL means 1×10⁵ cells/mL.

The use of the letter “Z” in the context of referring to pressure represents the number “10” as a multiplier such that, for example, 4Z dynes/cm² means 40 dynes/cm².

The use of the letter “X” or “x” in the context of referring to Young's Modulus or Effective Young's Modulus represents the number “20” as a multiplier such that, for example, 0.18× Pascals (Pa) means 3.6 Pascals.

The use of the letter “F” in the context of referring to frequency represents the number “1” as a multiplier such that, for example, 4F Hertz (Hz) means 4 Hertz.

The terms “Cell Line A” and “Cell Line B” as used herein refer to Chinese Hamster Ovary (CHO) cell lines that express proteins, such as antibodies.

The term “about” in the context of numerical values and ranges refers to values or ranges that approximate or are close to the recited values or ranges such that the inventions can perform, such as having a sought rate, amount, density, degree, increase, decrease, percentage, value or presence of a form, temperature or amount of time, as is apparent from the teachings contained herein. Thus, this term encompasses values beyond those simply resulting from systematic error. For example, “about” can signify values either above or below the stated value in a range of approx. +/−10% or more or less depending on the ability to perform. The preceding ranges are intended to be made clear by context, and no further limitation is implied.

Shear stress is defined as the fluid shear force acting tangentially to the cell surface and is expressed as force per unit area (dyne/cm2 or N/m2). Shear stress can be generated by agitated liquid moving past static cells, agitated cells moving through static liquid or by cells moving within an agitated, dynamic fluid environment. Fluid viscosity is typically measured in poise where 1 poise=1 dyne sec/cm2=100 centipoise (cp). The viscosity of water, one of the least viscous fluids known, is 0.01 cp. The viscosity of a typical suspension of eukaryotic cells in media is between 1.0 and 1.1 cp at a temperature of 25° C. Both density and temperature can affect the viscosity of a fluid.

A “nanoindenter,” as the term is used herein, shall refer to any controllable mechanical structure that may be used for characterizing a response of a solid material (such as biological tissue, for example) to application of a force over a region of the solid material.

As used herein, the terms “find sample”, “find-sample”, “find surface”, “find-surface”, and FS, refer to a procedure to determine the distance between the positioned probe and a target cell surface beneath the probe so that the displacement distance above each sample can be manually adjusted in response to each newly obtained FS distance.

As used herein, the terms “multiple increasing oscillation frequencies”, MIOF, “dynamic mechanical analysis”, and DMA, refer to a technique wherein direct stress is applied via targeted oscillations at select frequencies to characterize the viscoelastic properties of a material as a function of frequency. The stress is applied directly down onto the sample and the frequency recorded is a result of the rate at which the cantilever pressing on the sample moves up off the sample and down onto the sample.

As used herein, the terms “series of indendations”, SOI, and matrix scan, refer to a technique that comprises targeting different points on a single cell to determine the uniformity of the cell stiffness across one cell. Additionally, different points across adjacent cells can be targeted using this technique so that the uniformity of cell stiffness across adjacent cells of the same culture can be determined.

“Young's modulus” or YM is a mechanical property of a material, device or layer which refers to the ratio of stress to strain for a given substance. Young's modulus may be provided by the expression: E=stress/strain=(L0/ΔL) (F/A); where E is Young's modulus, L0 is the equilibrium length, ΔL is the length change under the applied stress, F is the force applied, and A is the area over which the force is applied. Young's modulus may also be expressed in terms of Lamé constants via the equation: E=μ3λ+2μλ+μ, where λ and μ are Lamé constants.

“Effective Young's Modulus” or EYM is the Young's Modulus that does not incorporate the Poisson ratio. The Poisson ratio considers the possible outward compression of a sample in a perpendicular direction in response to an indentation.

The storage modulus (E′) in viscoelastic materials, such as, for example, cells, measures the stored energy, representing the elastic portion. The loss modulus (E″) in viscoelastic materials measures the energy dissipated as heat, representing the viscous portion. The ratio of the loss modulus to storage modulus in a viscoelastic material is defined as the tan delta, which provides a measure of damping in the material. Tan delta can be expressed via the equation: tan delta=E″/E′. A tan delta value greater than 1 indicates that, for example, a cell, is more viscous than elastic.

“Frequency” is the number of occurrences of a repeating event per unit of time. It is also occasionally referred to as temporal frequency to emphasize the contrast to spatial frequency, and ordinary frequency to emphasize the contrast to angular frequency. Frequency is measured in hertz (Hz).

As discussed herein, “adherent cells” and “adherent cell lines” refer to cells in which the primary cultures are attached to a solid support and thus are anchorage-dependent cells. “Suspension cells” or “suspension cell lines” refer to cells in which the cultures are suspended in liquid media and thus remain in the fluid media. Accordingly, the present disclosure additionally provides methods of producing a line of adherent cells from suspension cells.

“Fetal bovine serum” or “FBS” is derived from the blood drawn from a bovine fetus. FBS provides a comprehensive assortment of components ranging from growth factors, vital nutrient supplements, hormone and cell proliferation factors, electrolytes and enzymes with the collective goal of supporting cell growth and proliferation. A key component within FBS is a host of adhesion factors which promote the attachment of cells to an appropriate surface (Devireddy et al., 2019). Accordingly, in one aspect of the present inventions, flasks seeded with suspension cells are introduced to a chemically-defined culture medium supplemented with a concentration of fetal bovine serum (FBS). In some embodiments, the chemically-defined culture medium is supplemented with a concentration of about 0.5% Y, about 1% Y, about 1.5% Y, about 2% Y, about 3% Y, or about 4% Y, FBS. In preferred embodiments, the chemically-defined culture medium is supplemented with a concentration of about about 4% Y, FBS.

In adherent cell culture, “confluency” refers to the percentage of a culture dish surface that is covered by adherent cells. In certain aspects of the present disclosure, the cell culture is allowed to grow to an adherent/suspension-cell confluency of, for example, 70%, 71%, 72%, 73%, 74%, 75%, 76%, 77%, 78%, 79%, 80%, 81%, 82%, 83%, 84%, or 85% total confluency. In other aspects, the cell culture reaches a confluency of about 85%.

“Passaging cells” or “cell passage” as used herein may refer to the removal of cell culture medium and any suspended cell culture. In the methods disclosed herein, cell culture flasks are passaged after at least about 85% confluency to remove suspension cells.

“Phosphate-buffered saline” or “PBS” refers to an isotonic solution used widely in biological applications for its isotonic and non-toxic nature to most cells. In certain embodiments of the methods disclosed herein, after suspension cells are passaged to facilitate their removal from the flask, the flask is submerged in PBS to wash away excess media, non-viable cells, and toxic metabolites.

“Titer” refers to the measurement of the concentration of, for example, a target protein in the bioproduction process. Titer is the primary benchmark characterizing upstream manufacturing efficiency, with higher titers generally indicating that more desired product is manufactured using the same or less amount of fluid or filled bioreactor volume. Accordingly, the present disclosure provides processes of bioproduction optimization comprising quantifying the impact of shear stress on cells and using shear stress data to adjust the levels of shear force applied during bioproduction. In preferred embodiments, optimization results in an increase in product titer and yield.

In several aspects of the methods disclosed herein, the culturing of adherent cells from suspension cells lasts for at least 72 hours. In some aspects of the methods, the cells are passaged for about four, about five, or about six passages, such as for six passages.

In some aspects of the inventions, the density of adherent cells is at least 13.84D×10⁵ cells/mL after six passages.

The present disclosure also describes cells, such as CHO cells, produced by the methods disclosed herein.

All numerical limits and ranges set forth herein include all numbers or values thereabout or there between of the numbers of the range or limit. The ranges and limits described herein expressly denominate and set forth all integers, decimals and fractional values defined and encompassed by the range or limit. The ranges and limits described herein expressly denominate and set forth all integers, decimals and fractional values defined and encompassed by the range or limit. Thus, a recitation of ranges of values herein are merely intended to serve as a shorthand method of referring individually to each separate value falling within the range, unless otherwise indicated herein, and each separate value is incorporated into the specification as if it were individually recited herein.

Further details of the disclosed methods and systems are provided below.

### II. Measuring the Effects of Shear Stress

In some aspects of the inventions, the cells utilized in the methods disclosed herein to quantify the impact of shear stress on cells are mammalian cells. In in further aspects of the inventions, the mammalian cells include Chinese Hamster Ovary (CHO) cells, Baby hamster kidney (BHK) cells, Human embryonic kidney 293 (HEK293) cells, HeLa cells, per.c6 cells, nonsecreting murine myeloma (NSo) cells, and Sp2/0 murine myeloma cells. Hydrophobic surfactants are often utilized in industry to mitigate the impact of stress on CHO cells. Antifoam is typically added to bioreactions to alleviate reduced gas transfer rates and cell confinement in the foam layer, offering cells protection from the bubbles that rupture in the foam (Ritacco, F. V., Wu, Y. and Khetan, A. (2018), Cell culture media for recombinant protein expression in Chinese hamster ovary (CHO) cells: History, key components, and optimization strategies. Biotechnol Progress, 34: 1407-1426. https://doi.org/10.1002/btpr.2706). However, polydimethylsiloxane-based antifoam or simethicone have notably been documented to increase the sensitivity of CHO cells to shear forces (Wang J., Shah N., Walther J., Lu J., Johnson T., Ren Y., Mclarty J. Methods for Improving Cell Viability in a Production Bioreactor. Genzyme Corporation (Cambridge, Mass., US) (2019). https://www.freepatentsonline.com/y2019/0285617.html). Other shear-protective additives such as Poloxamer-188 (P-188) and Pluronic F-68 (PF-68) can be introduced to the culture media, where suitable surfactant concentrations for the cell line, the media composition and process parameters should be considered (Sieck J. Addressing Shear Stress in Bioreactors. (2017) https://cellculturedish.com/addressing-shear-stress-bioreactors/. Accessed Sep. 23, 2022). P-188 has a high hydrophilic-lipophilic balance—determined mostly by the content of two hydrophilic poly(ethylene oxide) side chains—allowing it to act at bubble-cell interfaces to mitigate bubble-bursting stress during sparging (Chang D., Fox R., Hicks E., Ferguson R., Chang K., Osborne D., Hu W., Velev O. D. Investigation of interfacial properties of pure and mixed poloxamers for surfactant-mediated shear protection of mammalian cells. Colloids and Surfaces B: Biointerfaces, Volume 156. 2017. Pages 358 365. ISSN 0927-7765, https://doi.org/10.1016/j.colsurfb.2017.05.040). PF-68 also reduces hydrodynamic-damage to cells from bubble entrainment, through a protective layer around the cell membrane to enhance membrane integrity (Hu, W., Berdugo, C., & Chalmers, J. J. (2011). The potential of hydrodynamic damage to animal cells of industrial relevance: current understanding. Cytotechnology, 63(5), 445-460. https://doi.org/10.1007/s10616-011-9368-3), (Ritacco et al., 2018). Many approaches aim to optimize surfactant functionality, such as in the gradual transition in industry towards utilising serum-free, chemically defined medium. Serum medium has demonstrated a protective property against shear stress on animal cells, when compared to the absence of such protection in serum-free culture (Cynthia B, Elias T, Rajiv B. Desai T, Milind S. Patole, Jyeshtharaj B., Joshi T and Raghunath A Mashelkar. Turbulent Shear Stress—Effect On Mammalian Cell Culture And Measurement Using Laser Doppler Anemometer. Chemical Engineering Science, Vol. 50, no. 15. 1995.). For compensation, shear-protective additives have been efficiently integrated alongside serum-free media for use in CHO-dependent industrial bioprocesses (Li W., Fan Z., Lin Y. and Wang T. Y. Serum-Free Medium for Recombinant Protein Expression in Chinese Hamster Ovary Cells. Frontiers in Bioengineering and Biotechnology. 2021. https://www.frontiersin.org/articles/10.3389/fbioe.20 21.646363/full). Further optimisations have seen attempts to comprehend a common phenomenon of lot variability with P-188 (Peng, H., Ali, A., Lanan, M., Hughes, E., Wiltberger, K., Guan, B., Prajapati, S. and Hu, W. (2016), Mechanism investigation for poloxamer 188 raw material variation in cell culture. Biotechnol Progress, 32: 767-775. https://doi.org/10.1002/ btpr.2268), as well as managing concentration adjustments of PF-68 and antifoam to retain optimal oxygen transfer (Ritacco et al., 2018). These supplements rectify issues of cell sensitivity to shear, enabling productivity and consistency in bioproduction to be realized.

A. Chinese Hamster Ovary Cell Line Properties and Culture Characteristics

One distinct contributing factor into a cell line's tolerance to shear stress may involve how the cells are anchored in their growth conditions. Cells that are anchorage-independent are referred to as suspension cultures, as they are suspended freely in the medium. This is the typical growth conditions of CHO cells in industrial stirred tank bioreactor processes, due to their ease-of-use in scale-up operations (Rossi G. The design of bioreactors. Hydrometallurgy. 2001, 59 (2-3): 217-231). Suspended CHO cells can tolerate high degrees of agitation in a bioreactor; however, it has been observed that this tolerance is eradicated if bubble entrainment is also introduced (Hu et al., 2011). Adherent CHO cells on the other hand are dependent on a microcarrier environment in which they attach to and grow. Therefore, if agitation were to cause these cells to become detached from their microcarrier, they may become non-viable and prone to the potential devastating effects of hydrodynamic forces outside their comfort zone (Hu et al., 2011). As a result, cells in suspension can tolerate hydrodynamic conditions more than adherent cells that depend on surface-attachment. Furthermore, adherent CHO cells in particular are also known to be more sensitive to shear stress even when attached to their microcarrier environment, as they are not free to alter their orientation to alleviate any focused hydrodynamic stress on their membrane in the same way that freely-suspended CHO cells can (Goh S. “Micro-bioreactor Design for Chinese Hamster Ovary Cells.” Department of Materials Science and Engineering. Massachusetts Institute of Technology. (2013). https://core.ac.uk/download/pdf/18321479.pdf). Due to these outlined differences characterizing anchorage dependency, any adaptation of a CHO cell line from suspension to adherent—or vice versa—should acknowledge the potential for changes to their intrinsic shear-sensitivity that may not be possible to measure with a single instrument. Accordingly, in one embodiment of the present inventions, the methods comprise the use of CHO cells. In further embodiments, the CHO cells are suspension cells. In yet further embodiments, the suspension CHO cells are immobilized using a cell and tissue adhesive. In some embodiments, the cell and tissue adhesive is Cell-Tak.

B. Shear Stress

The impact of any degree of hydrodynamic stress can be segregated into two distinct categories: namely, lethal and sublethal effects on these cells. The lethal effects induced by shear stress include apoptotic and necrotic cell death, with a resulting decrease in cellular viability. It has been documented that the prevalence of shear-induced CHO lethality increased at the same time that cell viability decreased in the overall population (Godoy-Silva R., Chalmers J. J., Casnocha S. A, Bass L. A, Ma N. Physiological responses of CHO cells to repetitive hydrodynamic stress. Biotechnology and Bioengineering Vol. 103, Issue 6. (2009). https://doi.org/10.1002/bit.22339). This loss of structural integrity is central to reduced viability within the total cell count. However, the difference between necrosis being a ‘forced’ cell death and apoptosis being purposefully initiated by the cells through internal and external stress signals may influence the outcome of the inflicted shear stress (Fink, S. L., & Cookson, B. T. (2005). Apoptosis, pyroptosis, and necrosis: mechanistic description of dead and dying eukaryotic cells. Infection and immunity, 73(4), 1907-1916. https://doi.org/10.1128/IAI.73.4.1907-1916.2005). Namely, both apoptosis and necrosis are characterized by different morphological alterations in the cell structure, where cell death ultimately culminates in a cessation of protein production (Fink and Cookson, 2005). One publication determined that an abrupt, intense liquid-flow pressure on an adherent CHO K1 cell line could induce necrosis (Gregoriades N, Clay J, Ma N, Koelling K, Chalmers J J. Cell damage of microcarrier cultures as a function of local energy dissipation created by a rapid extensional flow. Biotechnol Bioeng. 2000;69:171-182. doi: 10.1002/(SICI)1097-0290(20000720)69:2<171::AID-BIT6>3.0.CO;2-C.). Another study that reported cell rupture of a suspended version of this CHO K1 cell line interestingly noted that the glucose and lactate cell metabolism were unaffected (Godoy-Silva et al., 2009). Accordingly, the present disclosure provides processes of bioproduction optimization comprising quantifying the impact of shear stress on cells and using shear stress data to adjust the levels of shear force applied during bioproduction. In certain preferred embodiments, optimization results in an increase in cell viability. In several embodiments, cell viability is measured by a bioanalyzer using a trypsin blue exclusion method.

In contrast to these lethal scenarios, less-intensive hydrodynamic forces have been documented as inducing sub-lethal adversities on many aspects of CHO cells. Hydrodynamic pressures inflicted on their membranes have the potential to indirectly alter the interior cytoskeletal framework, provoking sub-lethal ramifications on the cells. The cytoskeleton extends throughout the cytoplasm and consists mainly of three elements: actin filaments (microfilaments), intermediate filaments and microtubules. Published literature investigating how shear stress alters the cytoskeletal structure of CHO cells in particular is generally scarce. However, there are some detailed examples highlighting the importance of monitoring its impact on cellular structure and function. For instance, upregulated actin filaments in a suspension-adapted CHO-SA cell line were claimed to enhance the cells' survival chances from agitation-induced shear stress (Walther, C. G., Whitfield, R. & James, D. C. Importance of Interaction between Integrin and Actin Cytoskeleton in Suspension Adaptation of CHO cells. Appl Biochem Biotechnol 178, 1286 1302 (2016). https://doi.org/10.1007/s12010-015-1945-z). This report also discusses the occurrence of actin filaments interacting with integrin transmembrane receptor proteins on the CHO cells, providing a link between the interior cytoskeleton and the extracellular matrix, where the effects of shear stress could reverberate to the interior structure. Integrins have been documented as mediating attachment between cells and a fibronectin glycoprotein, a coating which is often used in adherent cell treatment to anchor cells to a surface (Wu C, Bauer J S, Juliano R L, McDonald J A. The alpha 5 beta 1 integrin fibronectin receptor, but not the alpha 5 cytoplasmic domain, functions in an early and essential step in fibronectin matrix assembly. J Biol Chem. 1993 Oct. 15;268(29):21883-8. PMID: 7691819.). There is a potential avenue for future research investigating how adherent or suspension CHO cells differ in their tolerance to shear stress specifically as a result of their alternate cytoskeleton structures. For example, an increase in shear stress rates on CHO cells has been shown to amplify the cell's adhesive properties through an integrin isoform (Vijayan K. V., Huang T. C., Liu Y., Bernardo A., Dong J., Goldschmidt-Clermont P. J., B. Rita Alevriadou, Bray P. F. Shear stress augments the enhanced adhesive phenotype of cells expressing the Pro33 isoform of integrin β3. FEBS Letters. Volume 540, Issues 1-3. 2003. Pages 41-46. ISSN 0014-5793. https://doi.org/10.1016/S0014-5793(03)00170-4.). Furthermore, a report on actin reorganization from fibrils to spherical sheaths when CHO cells were adapted from an adherent to suspension cell line (Walther et al., 2016) could indicate that these morphological changes may not have the exact same sensitivity to hydrodynamic forces.

In a recent study, the regulation of actin expression in the cytoskeleton of CHO cells has been associated with their productivity (Pourcel, L., Buron, F., Arib, G., Le Fourn, V., Regamey, A., Bodenmann, I., Girod, P. A., & Mermod, N. (2020). Influence of cytoskeleton organization on recombinant protein expression by CHO cells. Biotechnology and bioengineering, 117(4), 1117-1126. https://doi.org/10.1002/bit.27277). As shear stress may impact this structure and thus, the productivity of the cell line, this aspect should be considered seriously in any process to maintain the desired CQAs. In high-productivity CHO clones, the actin gene ‘ACTC1’ was found to be overexpressed, with the authors linking this high expression directly to the quantified improvement in CHO cell productivity (Pourcel et al., 2020). Additionally, the authors report that a low level of actin polymerization coincided with higher productivity. In their concluding remarks, they propose that a guanosine triphosphate GTPase-activating protein known as TAGAP could improve cell proliferation through mediating actin-integrin signaling. With the actin-integrin relationship potentially playing a role in proliferation—as well as their aforementioned roles under shear stress—this phenomenon demonstrates an intriguing, untapped field.

Actin upregulation in CHO cells is of further interest as it has been reported to diminish the levels of toxic lactate by-products being generated during metabolic activity (Pourcel et al., 2020). Interestingly, another previously cited paper found that when studying the impact of repeated shear stress on CHO cells, there was no significant difference in glucose utilisation and lactate production between the tests and the control (Godoy-Silva et al., 2009). This area of metabolic research deserves further investigation, especially as a previously-mentioned citation (Fan, Y., Jimenez Del Val, I., Muller, C., Wagtberg Sen, J., Rasmussen, S. K., Kontoravdi, C., Weilguny, D. and Andersen, M. R. (2015), Amino acid and glucose metabolism in fed-batch CHO cell culture affects antibody production and glycosylation. Biotechnol. Bioeng., 112: 521-535. https://doi.org/10.1002/bit.25450) discusses how lactate is detrimental to CHO cell growth and may increase its susceptibility to shear stress. Therefore, metabolic substrates and by-products may have a noteworthy influence on CHO cellular structure, integrity and hydrodynamic stress-tolerance.

A significant potential threat to product integrity is how shear stress impacts the CQAs of the protein produced in the bioreaction. Glycosylation is one of the most closely monitored CQAs in protein production, particularly in the production of therapeutic monoclonal antibodies using industrial CHO cell lines. Shear stress has been shown to have a physical impact on glycosylation efficiency, with one study demonstrating that hydrodynamic stress above 0.005 Nm-2 altered the endoplasmic reticulum (ER) of CHO cells (Keane J. T., Ryan D., Gray P. P. Effect of shear stress on expression of a recombinant protein by Chinese hamster ovary cells Biotechnol. Bioeng., 81 (2003), pp. 211-220

https://doi.org/10.1002/bit.10472), the organelle where glycan precursor assembly and initial modifications are performed (Schoberer J, Shin Y J, Vavra U, Veit C, Strasser R. Analysis of Protein Glycosylation in the ER. Methods Mol Biol. 2018;1691:205-222. doi: 10.1007/978-1-4939-7389-7_16. PMID: 29043680; PMCID: PMC7039702.). This is supported by another study, where the predominantly observed physiological impact of shear on CHO cells was a change in glycosylation pattern attributed to the repeated deformation of the ER (Godoy-Silva et al., 2009). Shear stress can also impact the timing of intracellular CHO cell trafficking and thus influence the glyco-profile. According to one study, a recombinant tissue-type plasminogen activator protein synthesized by these cells had a reduced residence time in the ER (Senger, R. S. and Karim, M. N. (2003), Effect of Shear Stress on Intrinsic CHO Culture State and Glycosylation of Recombinant Tissue-Type Plasminogen Activator Protein. Biotechnol Progress, 19: 1199-1209. https://doi.org/10.1021/bp025715f). They observed that shear stress correlated with an increased protein synthesis as a protective response followed by a restricted glyco-enzyme access, highlighting the serious impact that shear stress could have on product quality. Accordingly, a process of bioproduction optimization is disclosed herein, comprising the steps of quantifying the impact of shear stress on cells and using shear stress data to adjust the levels of shear force applied during bioproduction. In preferred embodiments, optimization results in an increase in product quality. In further embodiments, product quality is determined by glycosylation efficiency. In yet further embodiments, glycosylation efficiency is measured by chromatographic methods.

1. Inducing Shear Stress

a. Mechanical Agitation

Upstream bioproduction processes that require mechanical agitation either for dispersing oxygen to cells within the medium, promoting heat transfer through convection or maintaining cells in suspension must be carefully monitored and controlled to maintain cellular integrity (Nair A. J. Introduction to Biotechnology and Genetic Engineering (Principles of Biotechnology), Infinity Science Press LLC. Laxmi Publications. 2008. ISBN: 978-1-934105-16-2). General apprehension over harming CHO cells prone to high fluidic force damage may develop into a reluctance to operate at optimal agitation conditions. The flow of bulk liquid in a bioreactor is largely controlled by the mechanical agitation output, as well as the choice of impeller that ultimately generates the flow pattern (Rossi, 2001). Turbine impellers can induce excessive shear rates as a consequence of the high radial flow and subsequent longitudinal and tangential flows that are generated during its efficient mixing (Lebranchu A., Delaunay S., Marchal P., Blanchard F., Pacaud S., Fick M., Olmos E. Impact of shear stress and impeller design on the production of biogas in anaerobic digesters, Bioresource Technology. Volume 245, Part A. (2017). Pages 1139-1147. https://doi.org/10.1016/j.biortech.2017.07.113). Rushton turbine impellers have been documented to elicit shear stress on sensitive CHO cells, whereas pitch-bladed impellers are ideal for gentle mixing of these shear-sensitive cells (Mirro R. and Voll K. Which Impeller Is Right For Your Cell Line? BioProcess International. 2021.). Rushton turbine impellers can be combined with pitch-blades to reduce overall shear stress, while providing efficient mass transfer in the bioreactor (Karimi, A., Golbabaei, F., Mehrnia, M. R. et al. Oxygen mass transfer in a stirred tank bioreactor using different impeller configurations for environmental purposes. J Environ Health Sci Engineer 10, 6 (2013). https://doi.org/10.1186/1735-2746-10-6). Paddle impellers—along with the installation of baffles—can be employed to provoke mild agitation and overcome CHO cell-sensitivity to shear fluid force (Nair, 2008), (Mirro and Voll, 2021). The different laminar and turbulent flows that are generated by the impeller movement through the liquid can have a range of impacts on the cellular integrity; from minor to major morphological alterations, to the destruction of the whole cell (Mollet M., Godoy-Silva R., Berdugo C., Chalmers J. J. Acute hydrodynamic forces and apoptosis: A complex question. Biotechnology and Bioengineering. Vol. 98, Issue 4. 2007. https://doi.org/10.1002/bit.21476). Thus, it is important to note when evaluating process conditions that the tolerance of these cells to higher levels of mechanical agitation is dependent on the degree of hydrodynamic force generated by the agitation, as well as the characteristic sensitivity of CHO cells to this force (Godoy-Silva et al., 2009).

Cells could be introduced to hydrodynamic forces generated by a dedicated fluidic pump system. In a fluidic pump system, samples can be prepared by seeding cells in adherent conditions into the interior of appropriate slides to achieve the desired flowrate. Slide choices facilitating various interior volumes can be combined with a variety of connected tubing-system sizes to generate a vast range of possible shear conditions. For example, the different channel volumes in these slides cause the flow of liquid to pass through at different rates, despite the fact that the actual base surface area that adherent cells reside on is the same for all slides. The tubing associated with the pump apparatus can be connected to the slides at two channels on the closed-top surface, so that the pump can push the uncultured media through the tubing and into the slide's interior. This forms an ideal perfusion system, circling a controlled hydrodynamic flow around the system multiple times and particularly over the prepared adherent cells immobilized inside the slides. This unidirectional flow can impact adhered cells by exerting a force parallel to the surface of the fixated cell (Wang, Lu & Wu, Shuai & Fan, Yubo & Dunne, Nicholas & Li, Xiaoming. (2019). Biomechanical studies on biomaterial degradation and co-cultured cells: mechanisms, potential applications, challenges and prospects. Journal of Materials Chemistry B. 7. 10.1039/C9TB01539F.). A force exerted across a particular parallel surface-area on cells induces a shear stress, which depending on the extremity of the stress has the potential to impose lethal or sub-lethal deformation with potential lasting impact on their intrinsic visco-elastic properties (Kim L., Toh Y. C., Voldman J. and Yu H. A practical guide to microfluidic perfusion culture of adherent mammalian cells. The Royal Society of Chemistry, 7, 681-694. (2007). https://www.rle.mit.edu/biomicro/documents/lykim_LOC2007.pdf). Accordingly, in some embodiments of the inventions, the forces that cause shear stress to cells are generated by a fluidic pump system.

Agitation of cells to induce shear stress can also be initiated via shake flask or bioreactor agitation. Shake flask agitation can be initiated by seeding cells in a shake flask, followed by placing the flask on a rocker. The rocker can then be set to various revolutions-per-minute (rpm). Bioreactor agitation can be caused by an internal agitator or aerator. Accordingly, in some embodiments of the inventions, the forces that cause shear stress to cells are generated by shake flask agitation. In other embodiments of the inventions, the forces that cause shear stress to cells are generated by bioreactor agitation.

b. Bubble Destabilization

There is substantial evidence to suggest that CHO cells are prone to detrimental hydrodynamic stresses induced by the destabilization of oxygen gas-containing bubbles. One study details the phenomenon referred to as ‘bubble entrainment,’ in which bubbles become trapped in regions of turbulent flow created by mechanical agitation or aeration through sparging (Hu et al., 2011). Bubbles that become unstable may burst in the bioreaction medium, generating a force that can damage animal cells (including CHO), particularly due to the absence of a protective cell wall (Nair, 2008). Researchers have observed that CHO cells were able to withstand higher degrees of agitation in an industrial bioreactor when bubble entrainment was mitigated (Li F, Hashimura Y, Pendleton R, Harms J, Collins E, Lee B. Biotechnol Prog. 2006 May-Jun; 22(3):696-703.A systematic approach for scale-down model development and characterization of commercial cell culture processes.). This was achieved as the higher-implemented agitation speed reduced the aeration flow rate from the sparger. This demonstrates a parameter that can be adjusted to reduce shear stress, while maintaining the same optimal oxygen transfer rate to cells. While both agitation and aeration processes can be manipulated in an attempt to mitigate their additive devastation on cellular integrity, research has indicated that lowering the agitation power may actually contribute to bubble rupture and subsequent cellular damage (Ma N, Chalmers J J, Auniņš J G, Zhou W and Xie L (2004). Quantitative Studies of Cell Bubble Interactions and Cell Damage at Different Pluronic F-68 and Cell Concentrations. Biotechnology Progress. 20: 1183-1191.).

C. Measuring Shear Stress

In order to assess the impact of hydrodynamic stress on production CHO cell lines, it is imperative to perform appropriate quantification strategies to generate informative data. A range of techniques are outlined in the literature, which focus on different biomarkers to quantify the induced shear stress. Lactate dehydrogenase (LDH) assays have been used in a range of cytotoxicity studies over the years, namely the study of lethality in response to shear stress (Kaja, S., Payne, A. J., Naumchuk, Y., & Koulen, P. (2017). Quantification of Lactate Dehydrogenase for Cell Viability Testing Using Cell Lines and Primary Cultured Astrocytes. Current protocols in toxicology, 72, 2.26.1-2.26.10. https://doi.org/10.1002/cptx.21). This assay detects the release of intracellular LDH from damaged non-viable cells resulting from lethal shear rates. Typical assays utilize components such as Water-Soluble Tetrazolium Salts—which interact with NADH generated by purposeful LDH conversion to pyruvate—to measure the fluorescence that is proportional to LDH release and thus, cell damage (Kaja et al., 2017). Energy dissipation rates (EDR) have also been used to measure hydrodynamic stresses on CHO cells, particularly sub-lethal impacts where glycosylation patterns greatly shifted at higher EDR's (Godoy-Silva et al., 2009). This study amongst others uses scale-down bioreactors to replicate production shear rates. Other investigations have used microfluidic devices to concentrate a controlled laminar perfusion flow for quantifying hydrodynamic stress and subsequent (sub)lethal impacts (Kim et al., 2007).

1. Nanoindentation

A laboratory approach to quantify the viscoelastic properties of cells experiencing differing rates of shear was executed with the use of a nanoindenter. Nanoindenters have the ability to measure the mechanical and physical properties of small samples with precision and accuracy. This measurement is typically performed by indenting the sample to a desired and controlled depth using a hard tip component, in order to determine the unknown physical properties of the sample being studied (Bull S. J. Nanoindentation of coatings. 2005 J. Phys. D: Appl. Phys. 38 R393.). A range of nanoindenters have been used in many applications over the last century, most widely for investigating a material's sensitivity to penetration by a controlled load force. The degree in which the indenter can penetrate with ease or difficulty can provide an insight into the hardness of a material (Bull, 2005). Accordingly, in preferred embodiments of the methods disclosed herein, the mechanical properties of cells at different stress levels is measured by nanoindentation using a nanoindenter as disclosed herein.

A nanoindenter has the potential capability of measuring the impact of hydrodynamic shear forces on culture samples positioned under a mounted inverted microscope. During operation, an optical probe that is attached to the nanoindenter head can be mechanically lowered from a known, pre-calibrated distance above a culture dish towards the surface of a sample. In several embodiments of the methods disclosed herein, the probe is lowered for a period of about two seconds. The probe incorporates a thin cantilever, which bends upon contact with a sample surface. Lowering of the nanoindenter head is known as ‘displacement’, from which the degree of cantilever bending is subtracted by the software to calculate the indentation that occurs on the sample. This indentation exerts a force known as the ‘load’ upon contact with a surface area. In some embodiments of the methods disclosed herein, the cantilever is in contact with the cell surface for about one second. In other embodiments, the cantilever is in contact with the cell surface for about six seconds. In some embodiments of the present disclosure, the methods comprise determining a cell's mechanical properties after one round of nanoindentation. In other embodiments of the present disclosure, the methods comprise determining a cell's mechanical properties after several round of nanoindentation, such as about two, about three, about four, about five, or about six rounds of nanoindentation. In one preferred embodiment, the methods comprise determining a cell's mechanical properties after about six rounds of nanoindentation. In a further preferred embodiment, each subsequent nanoindendation is placed about 2 μm from the preceding nanoindentation.

In certain other embodiments of the methods disclosed herein, the cantilever generates multiple increasing oscillation frequencies (MIOF) of about 1F Hz, about 2F Hz, about 4F Hz, and about 10F Hz when in contact with the cell surface for at least about six seconds. The stress is applied directly down onto the sample and the frequency recorded is a result of the rate at which the cantilever pressing on the sample moves up off the sample and down onto the sample. If indentations indicated visco-elasticity (time-dependence), an experiment of MIOF would shed more light on the different types of frequency-dependent Young's moduli values present (Yablon D. Confusion of moduli. Wiley Analytical Science, Microscopy and Scanning Probe Microscopy, (2017). https://analyticalscience.wiley.com/do/10.1002/micro. 2417). For instance, a measured response of stored energy would elucidate the storage modulus (E′) representing more elasticity, while a measured release of energy would indicate the loss modulus (E″) representing more viscosity (Yablon, 2017). Accordingly, in further embodiments of the methods disclosed herein, MIOF were set at about 1F Hz, about 2F Hz, about 4F Hz and about 10F Hz, with relaxation periods of about two seconds between each increased frequency.

In several embodiments, the probe is mechanically raised off the surface of the cell for a period of about two seconds.

Utilizing the values obtained from nanoindentation, the system also generates a load- indentation curve, which highlights the approach of the displacement downwards towards the sample, before measuring the loading and unloading of the indenting probe. Here, the graphical load-indentation curve at the point of indentation can allow the software to signify the sample stiffness or ‘Young's modulus’ (YM), which can be documented from multiple indentations to elucidate a sample's mechanical properties.

The YM is a general measurement of a sample's ability to store the energy created from an induced indentation (Jastrzebski, D. Nature and Properties of Engineering Materials (Wiley International ed.). John Wiley & Sons, Inc. (1959)). In simpler terms, it measures a sample's tolerance to particular indentations, where a sample with a lower recorded stiffness could indicate a greater susceptibility to stress and strain. This system also calculates the Poisson ratio, which considers the possible outward compression of a sample in a perpendicular direction in response to an indentation (Sokolnikoff, S., Mathematical theory of elasticity. Krieger, Malabar F L, second edition, (1983)). A derivative of the YM called the ‘Bulk Young's Modulus’ incorporates this ratio, whereas the Effective Young's Modulus (EYM) disregards this compression phenomenon in its results. These provide a variety of parameters which can be used in the assessment of a cell sample's stiffness throughout their encounter with shear stress. The YM could be a property of interest towards maintaining cell viability in manufacturing processes, which could experience many potential shear stress instigators at critical stages. In preferred embodiments of the present disclosure, the methods comprise determining cell stiffness by calculating Young's modulus (YM) and Effective Young's modulus (EYM) values. In some aspects of the inventions, the YM and EYM of cells after 24 hours of shear stress is less than about 50×Pa. In some aspects of the inventions, the YM and EYM of cells after 48 hours of shear stress is less than about 50×Pa. In some aspects of the inventions, the YM and EYM of cells after 72 hours of shear stress is greater than about 500×Pa.

All methods described herein can be performed in any suitable order unless otherwise indicated herein or otherwise clearly contradicted by context. The use of any and all examples, or exemplary language (for example, “such as”) provided herein, is intended merely to better illuminate the inventions and does not pose a limitation on the scope of the inventions unless otherwise claimed. No language in the specification should be construed as indicating any non-claimed element as essential to the practice of the inventions.

### EXAMPLES

### Example 1: Nanoindentation of Cells Inflicted with Shear Stress Conditions

Experimental Nanoindentation Techniques Chosen for Cells

A number of techniques were selected and modified appropriately to document the mechanical properties of prepared cells. Firstly, a ‘find-sample’ (FS) procedure to determine the distance between the positioned probe and a target cell surface beneath was implemented for the start of every new sample measurement. Next, a standard indentation procedure of 6 seconds was set-up with the ability to manually adjust the displacement distance above each sample, in response to each newly obtained FS distance. Additionally, this indentation procedure was extended to an overall 10 seconds, to further clarify the properties of a target cell. Namely, as elastic samples are time independent and viscoelastic samples are time dependent (Ozkaya N. et al.. Fundamentals of Biomechanics: Equilibrium, Motion, and Deformation. Springer Science+Business Media, LLC (2012). Pages 368-373. DOI 10.1007/978-1-4614-1150-5_15.), a notable change in the YM recorded between the two differently-timed indentations may elucidate the cell's mechanical properties. As a maximum indentation depth of 16% of the probe tip-radius and 10% of sample thickness has been recommended in the literature and by the manufacturer for nanoindentations, a depth management procedure to ensure this was controlled was implemented separately (Lin, D. C., Shreiber D. I., Dimitriadis E. K., Horkay F. “Spherical Indentation of Soft Matter beyond the Hertzian Regime: Numerical and Experimental Validation of Hyperelastic Models.” Biomechanics and Modeling in Mechanobiology 8, no. 5 (2009): 345-58. https://doi.org/10.100 7/s10237-008-0139-9). If indentations indicated visco-elasticity (time-dependence), an experiment of multiple increasing oscillation frequencies (MIOF) could shed more light on the different types of frequency-dependent Young's moduli values present (Yablon, 2017). For instance, a measured response of stored energy would elucidate the storage modulus (E′) representing more elasticity, while a measured release of energy would indicate the loss modulus (E″) representing more viscosity (Yablon, 2017). Finally, a series of indentations (SOI) aimed to target different points on a single cell was established to determine the uniformity of the YM across one cell.

Assessing the Compatibility of the Shear-Inducing Pump System with the Nanoindenter

As some adherent cells had been recovered from the shear-inducing pump system, a method of preparing these extracted cells for nanoindentation was assessed. Extracted Cell Line A and B cells were seeded onto petri dishes rather than T75 flasks, to allow the nanoindenter probe to gain direct access to a cell sample. As the lid on a petri dish had to be removed in this non-sterile testing environment for indentation, the sample was acknowledged as compromised and was discarded after use. Two types of petri dishes were immediately obtainable in the lab; a large glass petri dish and a smaller non-tissue culture-treated plastic petri dish. The glass petri dish was too large to position under the microscope and therefore incompatible with the mounted nanoindenter; however, it was still utilized for the purpose of comparison. As sample immobilization is imperative for accurate indentation results, both dishes were assessed to confirm their potential to facilitate cell adherence after 24 hours. For both cell lines after this timepoint, the adherent morphology was noted as present in large glass petri dishes, with only spherical cells identified in the smaller plastic dish. However, the spherical-shaped morphology should not be assumed to be wholly suspension. To confirm their characteristic non-adherence to this plastic dish, the sample was tilted, and all cells were seen to move with the media displacement, confirming non-adherence.

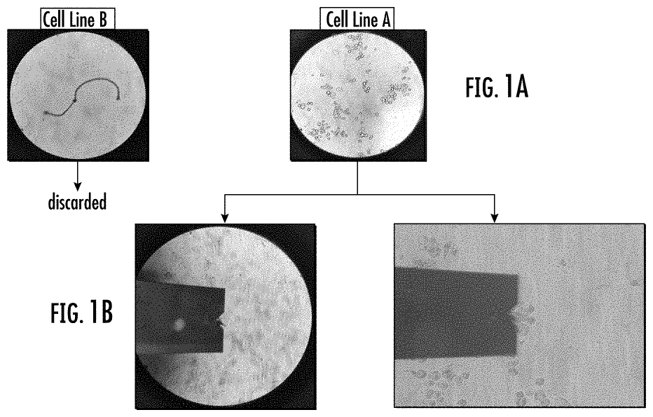

To accommodate the issues with the glass dish being too large and the plastic dish not facilitating adherence, a different approach was performed in which two naked glass slides were seeded with adherent Cell Line A and B cells respectively and submerged in media inside a large glass petri dish, so that they would remain sterile in an incubator over 72 hours. Then, this glass slide with cells anchored on its surface could be transferred to a smaller plastic dish suitable for nanoindentation. The large dish was chosen for the incubation period in order to compare any difference in adherence between cells on the glass slide or dish surface at the same density. After this timepoint, the glass slides were observed for both cell lines (FIG. 1). Cell Line A was noted as spherical with no elongation identified; however, upon tilting the dish the cells appeared to remain in-place. The glass petri dish area appeared to have more cell-mobility present, but anchorage was still identified. For Cell Line B, there was an immediate identification of discoloration and higher turbidity of the media, despite both dishes being seeded and incubated at the same conditions. The microscope indicated that there was contamination, through the visible presence of solely unexpected cell shapes and deformities (FIG. 1A). Therefore, only the Cell Line A sample was carefully transferred to the smaller dish to assess its suitability for nanoindentation. At this stage, a decision was made to not proceed with a nanoindentation attempt based on the following reasons surrounding how the sample had been prepared. While cell-anchorage was observable, a problematic number of suspended cells were latching onto the probe when lowered into the sample (FIG. 1B). This greatly disturbed an interferometer reading, which records a required optical wavelength path through the medium for the initial FS procedure on the targeted sample. It also posed a risk of damaging the sensitive cantilever and collecting unreliable indentation results. Tilting to confirm that a targeted spherical-shaped cell was anchored could only be performed when the probe was greatly raised above the sample. Positioning the lowered probe directly above an identified spherical, anchored cell repeatedly displaced the surrounding suspension patterns, making it difficult to identify the targeted cell under the microscope. Therefore, it was acknowledged that this methodology required revision in order to study cells experiencing stress.

Assessing a Cell and Tissue Adhesive as a Sample Preparation Solution for Nanoindentation

Difficulties with preparing and indenting the adherent cells highlighted a need to re-assess the current methodology. While obtaining smaller glass dishes could solve the issues with dish-size and surface-anchorage, a more cost-effective, forward-thinking approach was developed according to the inventions. Currently, the potential for suspension cells to re-orientate their position in the medium to accommodate any inflicted stress would make it difficult to document the direct impact of shear stress conditions on the stiffness of the cells (Goh, 2013). By ordering a stock of a cell and tissue adhesive (CTA) solution, such as Cell-Tak, both adherent and suspension cells could be immobilized on the plastic and glass dishes, as well as glass slides. This allowed for the possibility to resolve some issues preventing nanoindentation of adherent cells, as well as expanding the future capabilities of the nanoindenter to study immobilized suspension cells. With the plastic dishes being the ideal size for the microscope stage, the CTA solution was prepared by a neutralization step to active its adhesive properties and subsequently coated onto these dishes as a proof-of-concept.

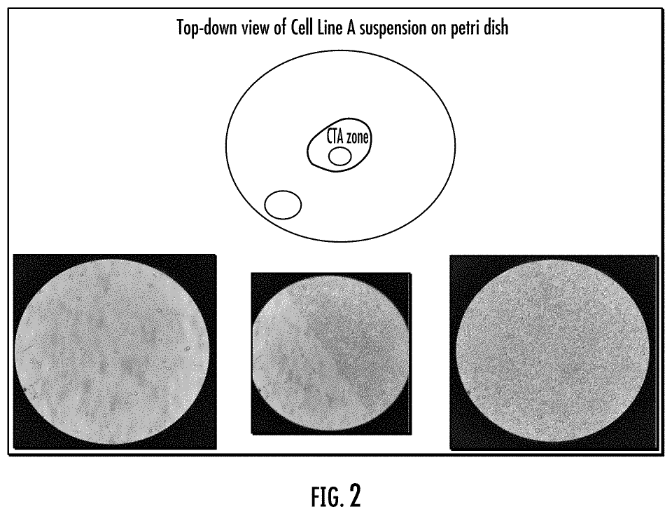

The proof-of-concept was tested with the original suspended form of Cell Line A, as a higher seeding density could be achieved in comparison with the current low yield of adherent cells extracted from the pump slides. A density of 51D×10⁵ cells/mL of suspension Cell Line A derived from a shake flask one day post-inoculation was seeded onto a plastic petri dish prepared with CTA coating. The objective was to determine whether the solution was optimally prepared and had a sufficient pH neutralization to activate the CTA and mediate cell-immobilization. After allowing cells to settle for ten minutes in the incubator, a microscope observation confirmed that a high cell density was present in the expected immobilization zone, whereas outside this zone indicated a low density of suspension cells (FIG. 2). To confirm immobilization, the dish was tilted, and cell mobility was only noted outside the coating zone. After removing the full media content, cells were predominantly retained within the CTA zone and mostly removed outside of it. Adding fresh media back to the plate did not appear to dislodge any of the immobilized cells within the CTA zone.

Nanoindentation of CTA-Immobilized Suspension Cells Retrieved from a Shake Flask over 72 Hours

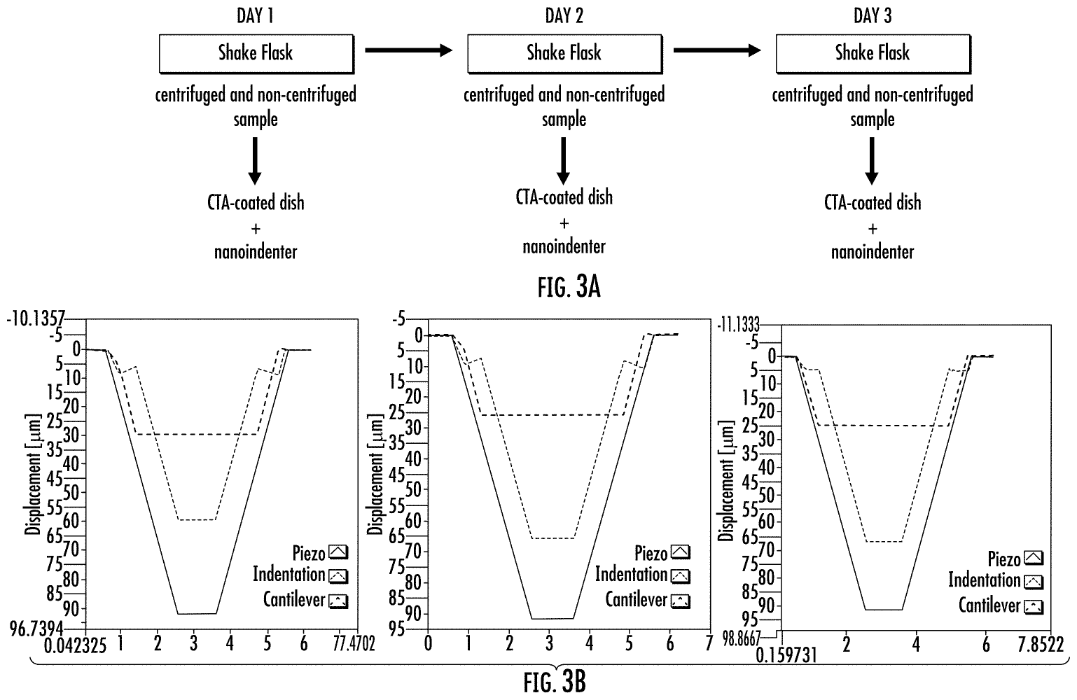

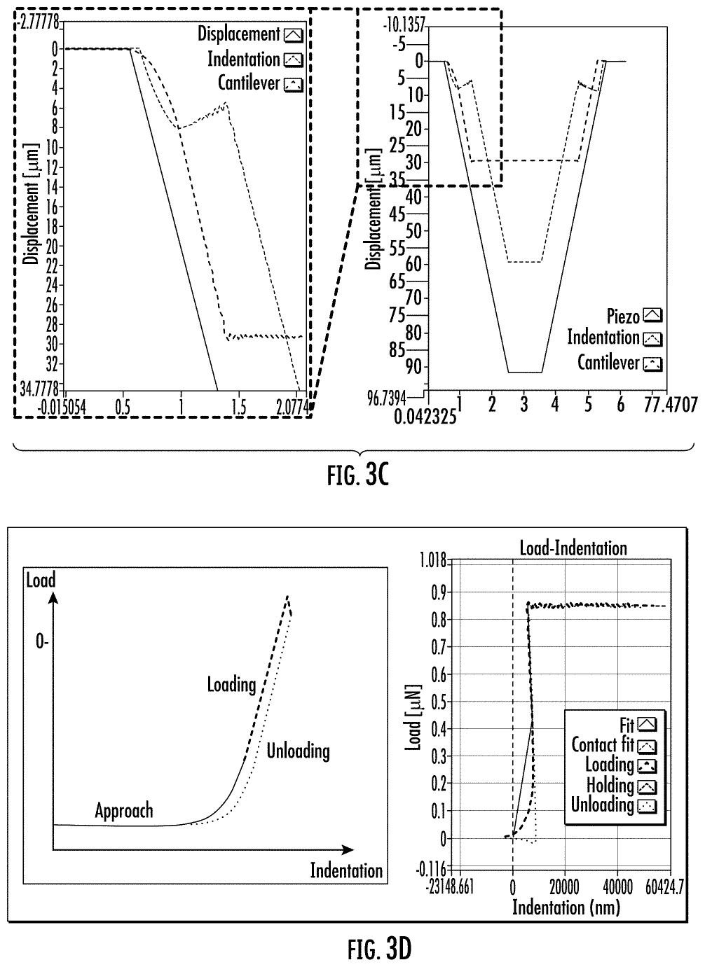

The plan to indent adherent cells from a stationary T75 flask was abandoned due to the lack of an appropriate yield of cells post-shear for comparison. Therefore, a comparison of suspension Cell Line A stiffness over the course of a shake flask agitation process was performed. In order to test the compatibility of this nanoindenter with suspension cells immobilized by the CTA solution, a stored vial of Cell Line A was thawed into a shake flask with samples subsequently seeded onto CTA-coated dishes. The aim of this study was to document any change in recorded cell stiffness data over the length of the 72-hour shake flask run (FIG. 3A). Shortly after the shake flask inoculation, a Day 0 sample of cells were withdrawn and immobilized for nanoindentation. This initial test displayed issues similar to the freely suspended nanoindentation attempt; cells that were not immobilized latched onto the probe again and as the opened dish was compromised, a build-up of suspended foreign contamination became visible over time. Furthermore, the first indentation test following the FS procedure generated a large background noise on the load recorded due to the unwanted attachment of suspension to the probe, prompting experimental cessation and revisions. The most important amendment was a five-fold increase in cell seeding-density, performed by centrifuging each sample withdrawn during Day 1-3 and resuspending it in a lower volume for high-density seeding onto dishes. However, it was acknowledged that centrifugation had the potential to introduce unwanted shear from centrifugal forces (Pembrey R S, Marshall K C, Schneider R P. Cell surface analysis techniques: What do cell preparation protocols do to cell surface properties?. Appl Environ Microbiol. 1999 July; 65(7):2877-94). Therefore, for each daily sample test, the normal, non-centrifuged sampling was also retrieved in an attempt to compare its suitability to the centrifuged sample at their respective VCD's over the three days of sample-withdrawal. Additionally, a PBS wash step was utilized to remove and mitigate interference from mobile cells.

Standard and Extended Indentations of CTA-Immobilized Suspension Cells

For Day 1-3 centrifuged samples, the initial FS technique before each sample-indentation experiment was executed with minimal issues. An initial indentation procedure to assess the successful interaction of the probe with each target cell surface produced a clean displacement-versus-time graph for each daily sample (FIG. 3B). The Day 1 standard indentation on an approx. seeding density of 120D×10⁵ cells/mL recorded a YM of 0.18×Pa and EYM of 0.24×Pa, with the following day's indentation over a higher density recording a lower YM and EYM of 0.1×Pa and 0.13×Pa respectively. This should indicate a decreased stiffness recorded on the Day 2 standard indentation. However, as the Day 3 sample recorded a higher 0.4×Pa YM and 0.54×Pa EYM, the software option to run the experiment on the same position was repeated two more times, displaying similar indentation curves but recording slightly lower YM and EYM. Targeting a different cell produced a YM and EYM of 3.8×Pa and 5.1×Pa respectively, a stark contrast to previous results. An assessment noted that for all samples, the bending of the cantilever occurred much sooner than anticipated when the probe began the FS-calculated displacement distance downwards towards each sample (FIG. 3C). This would indicate that the displacement approach downwards was immediately loading onto the sample, despite the FS procedure detecting the distance between the probe and the sample as a minimum of 90 μm. This immediate loading was confirmed by the load-indentation curves for all standard indentations (FIG. 3D).

Indentations with longer holding times on the surface of a target cell were also performed, with all recorded YM indicating a changed, more-robust stiffness to this increased timing, as expected of time-dependent viscoelastic samples (Ozkaya et al., 2012). However, a YM of 0.4×kPa recorded on a Day 3 target cell was much greater than the YM of 20.6×Pa and 25.7×Pa for Day 1 and 2 samples respectively. While a change in YM was expected, the large difference recorded here appeared to be unreliable, particularly with multiple indentations producing different values thus far. A depth management experiment on these same target cells was performed with the estimated average cell diameter by the bioanalzyer considered, to ensure that the indentation into target Cell Line A cells did not exceed the recommended depth. A conservative depth threshold was integrated and was suitable for all tests, as the standard deviation between the Day 1-3 average cell diameters was only 0.8 μm.

Multiple Increasing Oscillation Frequencies Inflicted on CTA-Immobilized Suspension Cells

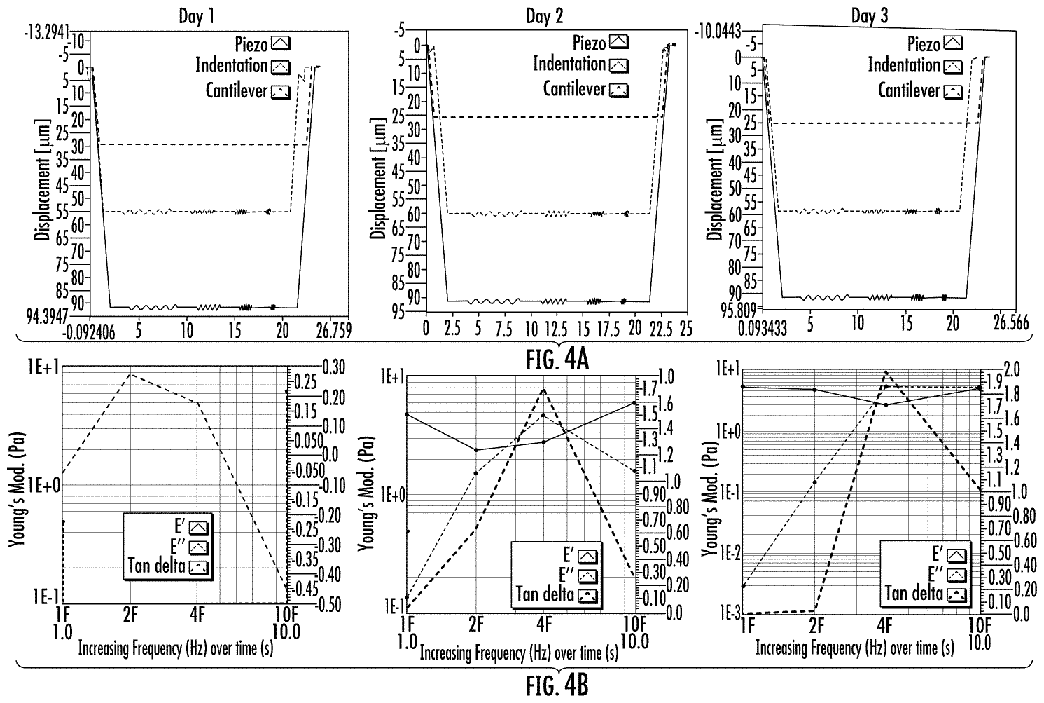

Introducing MIOF into the extended indentation length produced similar displacement-versus-time graphs showcasing the spaced-out, increasing frequencies over the surface of three samples (FIG. 4A). However, a graph of the storage modulus (E′) versus the loss modulus (E″) providing an insight into the viscoelasticity differed between the three samples (FIG. 4B). The Day 1 sample did not appear to efficiently record these moduli, however a tan delta value of the ratio between E″:E′ was calculated. A better interpretation can be found with the Day 2 sample data, which primarily indicated a dominance of elasticity of the sample over the frequencies of 1F, 2F and 10F Hz, with only the 4F Hz frequency displaying a dominance of the E″ representing viscosity. Intriguingly, the same trend was observed with the Day 3 sample, with both Day 2-3 tan delta curves displaying the same, overall characteristic viscoelasticity trend.

Series of Indentations on CTA-Immobilized Suspension Cells

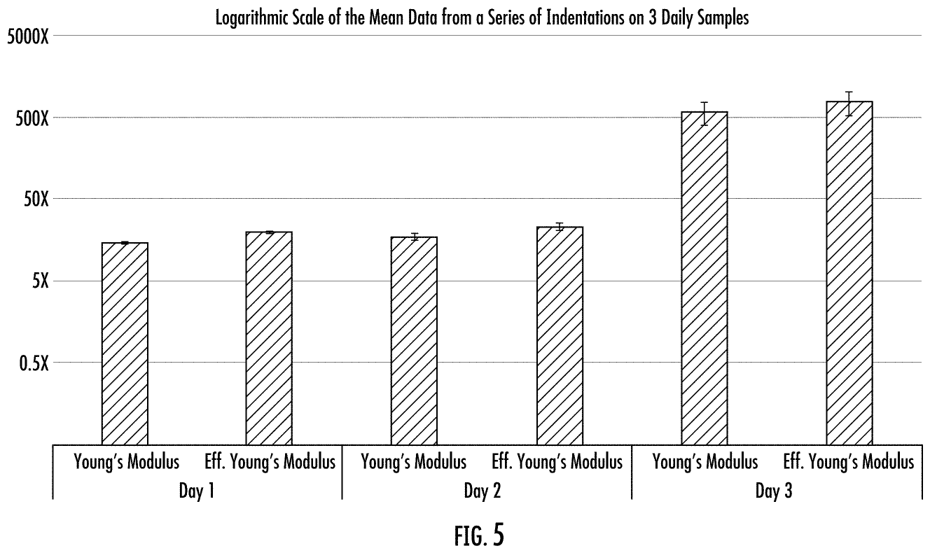

For the SOI experiments, six indentations were performed on each day's target cell. Bar charts produced by the software indicated that all six indentations pinpointing differently spaced and measured points of each cell successfully indented the samples. Therefore, the cantilever did not miss the cell surface by moving off the edge of the cell when changing pinpoints to indent over the predicted area. New bar charts were created with the obtained data to assess the uniformity of the mean sample-stiffness over the course of the 3-day shake flask process (FIG. 5). The data from the Day 1-2 target cells displayed similar means of YM and EYM, with the Day 1 sample having a smaller standard deviation from the mean. Both the YM and EYM of the Day 3 sample were recorded above 500×Pa, with a larger standard deviation from the mean. These large Day 3 values display a similar trend of increased values on Day 3 when compared to the YM and EYM obtained with the standard indentation for the same Day 3, indicating consistency within this third day's data, if not with the earlier two samples. Day 3 Pa values indicate that cells had a stiffer membrane on Day 3 than on Days 1 and 2. By Day 3, a density of 120D×10⁵ cells/mL was recorded for the uncentrifuged sample, matching the density of the Day 1 centrifuged sample tested. In theory, this density should be suitable based on the Day 1 compatibility, however the sample had been left for a few hours and began detaching from the CTA coating, preventing further study.

### Example 2: Generation of Adherent Cell Line A

Strategy to Generate Adherent Cells from Cell Line A

To support the adaptation of suspension cells to adherent growth—as well as their continued proliferation in this new cellular form—the chemically-defined culture medium was supplemented with fetal bovine serum (FBS). This serum provided a comprehensive assortment of components ranging from growth factors, vital nutrient supplements, hormone and cell proliferation factors, electrolytes and enzymes with the collective goal of supporting cell growth and proliferation. A key component within FBS is a host of adhesion factors which promote the attachment of cells to an appropriate surface (Devireddy L. R., Myers M., Screven R., Liu Z., Boxer L. and Ambrosio C. E. A serum-free medium formulation efficiently supports isolation and propagation of canine adipose-derived mesenchymal stem/stromal cells. PLoS One Journal, 2019; 14(2): e0210250. DOI: 10.1371/journal.pone.0210250). The presence of adhesion molecules in the serum presented an ideal avenue to begin the transition of suspension culture to adherent growth. From the moment that an established suspension Cell Line A vial was thawed, the cells were introduced to this serum to supply the necessary factors to begin this adaptation.

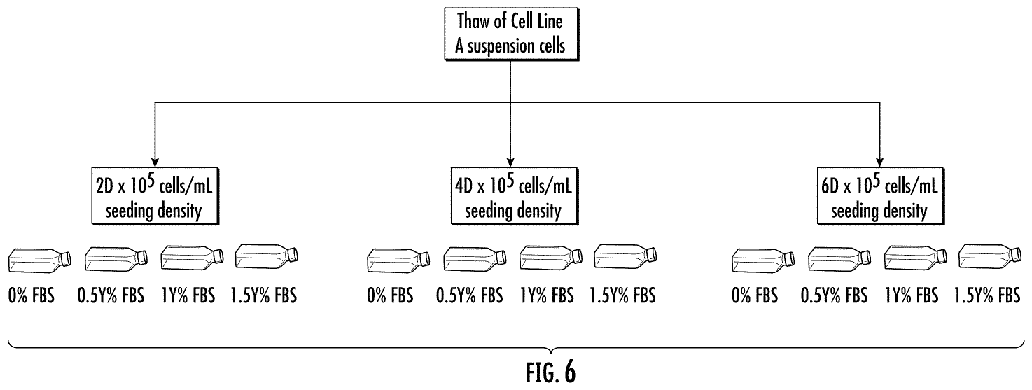

An initial test was established with alternate seeding densities of suspension cells of Cell Line A in T75 flasks, with a range of FBS concentrations supplemented in the chemically-defined medium (FIG. 6). Due to logistical constraints, the purpose of this initial experiment was to understand the growth, proliferation and development of any adherent cells over time, before proceeding to a more refined passaging protocol. The chosen initial seeding densities for Cell Line A were inspired by previous studies with Cell Line B, as both cell lines share similar properties. In this study, the optimal conditions for adherent Cell Line B growth had been established as 4D×10⁵ cells/mL supplied with 1Y% FBS. An upper and lower seeding density and FBS concentration around these previously-established optimal conditions for Cell Line B was applied to this initial Cell Line A study. Results were expected to be close—but different—to previous findings, as there were anticipated variances between both cell lines possibly centred around their growth in contrasting chemically-defined media formulations and their intrinsic genetic properties.

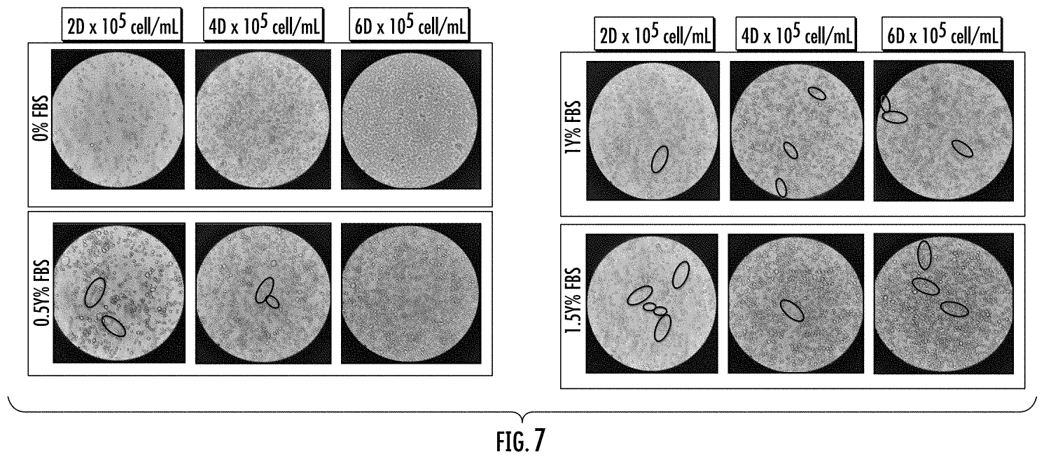

After seeding these T75 flasks, adherent cells were identified under a microscope by their elongated morphology, differentiated from the round, suspended cells present in the same flask (Abcam (n.d.) Cell Culture Guidelines. No date. Accessed on 10 Jul. 2021 at: https://www.abcam.com/ps/pdf/protocols/cell_culture.pdf). The flasks were particularly observed after a time period in which passaging would have been anticipated as necessary, in order to compare and contrast the growth and proliferation of the cells in each of the conditions at this critical point (FIG. 7). At this stage, most flasks were in excess of a total adherent/suspension-cell confluency of 70%, at which point it was decided to set a parameter of 70-85% total confluency as the indicator for imminent passaging required. The control—consisting of media absent of FBS—demonstrated a complete lack of the distinct cell morphology from the point of each seeding inoculation to the end of the experiment. In contrast, all other flasks containing FBS-supplemented media displayed the characteristic adherent morphology after 24 hours of growth. In particular, the results indicated that a gradual increase in adherent cell proliferation correlated with higher FBS concentrations. The seeding density of 4D×10⁵ cells/mL and 1Y% FBS media—mimicked from the previous adherent Cell Line B

Optimizing the Process of Generating Adherent Cells from Cell Line A

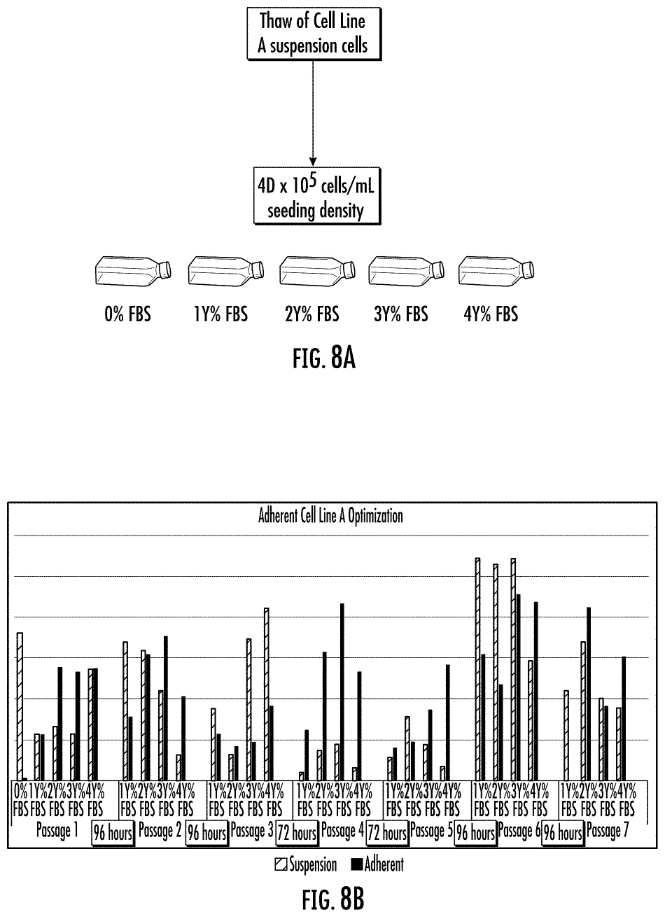

With an acquired understanding of the rate and efficiency of adherent Cell Line A generation, a more controlled and predictable timeline could be set-up to study which conditions were optimal for the continued proliferation of these cells after passaging. The optimal seeding density thus far (4D×10⁵ cells/mL) was chosen for more-efficient management of T75 flasks and to narrow down the optimal FBS conditions. As the previous Cell Line A study indicated that an increasing FBS concentration gradient led to more cell- anchorage, a wider range of FBS concentrations was investigated (FIG. 8A). The results of this particular study supported the extrapolation of FBS concentrations, which achieved increased cellular surface-attachment. Before the first passaging of the flasks was performed, a higher confluency of adherent cells in the 2Y%, 3Y% and 4Y% FBS concentrations were observed in comparison to the initial study's lower FBS tests. For the passaging protocol itself, a bioanalyzer was used to record the VCD of the suspension cells prior to passaging, so that it could be compared to the VCD of adherent cells retained during passaging (FIG. 8B). This motive is best explained by looking first at the control result during passaging, which was expected to have no adherent cells. In the absence of FBS (0%), the control's suspension VCD was high prior to passaging. During passaging, the culture media containing the suspension cells was removed and the surface area of the T75 flask was submerged with a thin layer of Phosphate Buffer Saline (PBS), in order to wash away any excess media, non-viable cells and toxic metabolites released (Segeritz, C. P., & Vallier, L. (2017). Cell Culture: Growing Cells as Model Systems In Vitro. Basic Science Methods for Clinical Researchers, 151-172. https://doi.org/10.1016/B978-0-12-803077-6.00009-6). PBS also aids in the efficient extraction of adherent cells from the flask, as the washing step allows a subsequent trypsin addition to focus on the detachment of remaining adherent cells rather than the break-down of culture proteins that would remain otherwise. Trypsin addition was used to hydrolyse cell-surface adhesion proteins facilitating surface-anchorage (Olsen J. V., Ong S., Mann M. Trypsin Cleaves Exclusively C-terminal to Arginine and Lysine Residues. Technology, Molecular & Cellular Proteomics 3:608-614 (2009). https://doi.org/10.1074/mcp.T400003-MCP200). In the case of the 0% FBS control conditions, trypsinization produced an extremely low yield of cells in stark contrast to the suspension recorded pre-passaging (FIG. 8B); therefore, the yield was assumed to be suspension cells retained from the PBS wash step. The control had to be discontinued, after an insufficient volume of resuspended culture was required to seed remaining cells back into a new T75 flask after passaging. It was concluded that this control demonstrated the requirement of FBS to generate adherent cells.

Trypsinization of cells across the range of FBS concentrations resulted in an irregular pattern of recorded suspension and adherent VCD's (FIG. 8B). The data indicated that for all four FBS concentrations tested, the suspension-to-adherent ratio was generally in favour of adherent growth after a 72-hour period. The low suspension VCD recorded after 72 hours (passage 4 and 5)—along with the generally higher yield of adherent cells—indicated that more cells had adapted over 72 hours from the initial suspended culture seeding density inoculated at the end of each passage. Passage 6 in particular highlighted the dominance of non-adherent cells, possibly due to a reduced anchorage over the extended time period. As this test also repeated the same seeding density and particularly the 1Y% FBS conditions seen in the previous attempt (FIG. 7), it gave a good indication of the reproducibility of comparative adherent growth observed under the microscope. Adherent growth at higher FBS concentrations also displayed a departure from previous adherent optimization studies, as 2Y% FBS led to uncontrollable growth of Cell Line B. For Cell Line A however, conditions up to 4Y% FBS all displayed increased adherence. After seven passages, the 3Y% FBS at this seeding density was chosen as the optimal conditions for adherent Cell Line A generation, due to its average yield and maximum single yield (passage 4 and 6) being the greatest.

### Example 3: Bioproduction Process Optimization

Cells are first subjected to shear forces, for example hydrodynamic forces, created by shake flask agitation or bioreactor agitation to inflict shear stress on the cells using methods described in Example 1. Then, the cells are sampled at various stages of cell expansion or bioproduction processes and subjected to nanoindentation to determine the viscoelastic nature of the cells after being subjected to shear stress. A find-sample technique, followed by a single indentation at two different lengths of time (seconds), are performed to determine if the Young's modulus is time dependent or not. A depth management procedure is performed to manage the indentation depth. Nanoindentation experiments to determine cell stiffness include multiple increasing oscillation frequencies (MIOF) and series of indentations (SOI) experiments. MIOF experiments indicate whether the viscoelastic nature of a cell is frequency-dependent. SOI experiments indicate whether the viscoelastic nature of a cell is uniform across the surface of a cell. Cells with higher degrees of stiffness are considered more resistant to shear stress than cells with lower degrees of stiffness, as determined by calculating Young's Modulus and Effective Young's Modulus.

When nanoindentation results indicate that cells are more susceptible to shear stress at certain levels of shear forces, the levels of shear forces generated by agitation may be adjusted to decrease the levels of shear stress inflicted on cells. Decreasing shear stress in cells will lead to an increase in cell viability throughout the bioproduction process. Decreasing shear stress will also lead to an increase in bioproduction product titer, yield, and quality, for example, glycosylation efficiency. Cell viability is measured by a bioanalyzer using a trypan blue exclusion method. Glycosylation efficiency and product titer and yield are measured by chromatographic methods.

When nanoindentation indicates that a certain cell or cell line is more resistant to shear stress at higher levels of shear forces, the cell or cell line is propagated and utilized in bioproduction processes.

### Example 4: Materials and Methods

Vial Thaw of a Suspension Cell Line A Cell Bank

Approximately 150 mL of Cell Line A media was warmed within the acceptable time period at the designated incubation temperature. A suspension Cell Line A vial was thawed in a water bath at a different designated temperature for several minutes within the acceptable time range. Thawed vials were pipetted into a designated volume for separating the cells from the freezing medium by centrifugation. The obtained pellet was resuspended in a fresh media volume identical to the original vial volume, before being directly seeded into an appropriate media volume in a T75 flask. A culture sample was later aspirated from this T75 flask to determine that the VCD was within the acceptable range, before the flask was returned to an incubator at the designated CO₂ and temperature values.

Passaging and Cell Culture Techniques

T75 flask confluency was observed every 24 hours under an inverted microscope, with an estimated total visual cell confluency of 70-85% used as an indicator to initiate passaging. Before passaging, a required amount of appropriate Cell Line A and Cell Line B media was warmed within the acceptable time period at the designated incubation temperature. Appropriate volumes of FBS and trypsin were thawed prior to experimentation. FBS concentrations for respective T75 flask passages were calculated based on the media required for multiple flasks. In a biological safety cabinet, spent media was removed from T75 flasks for VCD and viability sampling of suspension cells and subsequently discarded. The interior flask surface was washed with a designated volume of PBS and flooded with trypsin. Trypsin residue was removed, and flasks immediately incubated for 3 minutes. Bottom and sides of the T75 flask were tapped to dislodge cells. Remaining trypsin was neutralized with fresh media. Neutralized culture media was extracted for centrifugation to remove trypsin, with excess culture used for VCD and viability sampling of adherent cells. Pellets of centrifuged culture were resuspended in fresh media and seeded back to designated densities into fresh T75 flasks based on calculations of the VCD sampling of adherent cells recorded by the bioanalyzer. T75 flasks were returned to the incubator at the designated CO₂ and temperature values.

Hemacytometer Cell Counting

The ratio of cell suspension to trypan blue solution was prepared as a mixture with a dilution factor of 2. Cells were seeded into two separate grooves in the hemacytometer each with separate internal grids of four. Both grid counts required a cell count of viable cells between 80 and 200 cells. The final cell count for both groove areas were confirmed as being within 10% of each other for accuracy. The VCD and viability were determined through calculations incorporating the mean VCD counted, the dilution factor and the number of hemacytometer grids that cells were counted in.

Inflicting Shear Stress with the Fluidic Pump System

Appropriate volumes of Cell Line A and Cell Line B media was warmed within the acceptable time period at the designated incubation temperature. Adherent cells were extracted as part of the passaging process and seeded into Slide C at the required volume. Slide channels were covered with plastic caps and returned to the incubator at the designated CO₂ and temperature values for overnight adhering. The chosen Tubing A was placed in the same incubation conditions overnight to facilitate degassing of the tubes. Following incubation, four pump perfusion sets were mounted with Tubing A closed off to itself and two sets were topped up with respective Cell Line A and B media. Perfusion sets were connected to the pump hardware via cables and an air inlet tubing and inserted into the incubator. Pre-experimental equilibration of the system was set for ten minutes with a programmed bubble-removal step. Perfusion sets were disconnected from the pump and connected to respective Slide C slides in the biological safety cabinet, via clamped tubing. Perfusion sets were returned to the incubator. A pre-equilibration period of 0.5Z dynes/cm² for 2 hours and experimental period of Z dynes/cm² was performed initially for 26 hours on adherent cells obtained from the second passage, seeding 4D×10⁵ cells/mL with 9% FBS media into Slide C. Both shear rates were halved in later experimentation, using cells from passage 6 and 10 at seeding densities of 4D×10⁵ cells/mL and 60D×10⁵ cells/mL respectively. The 60D×10⁵ cells/mL VCD was acquired through centrifugation of adherent cells retained from passage 10, resuspended to achieve the appropriate density.

CTA Solution Preparation and Cell Seeding

A prepared 0.1 M sodium bicarbonate (84 g/mol), pH 8.0 solution was mixed with prepared 1N NaOH (40 g/mol) to aid in reaching a pH between 6.5-8.0, allowing the mixed CTA solution's adhesive properties to be activated. The supplier recommendations were followed for the ratio of CTA solution to each base to formulate the desired concentration. A small volume of CTA solution was pipetted directly onto the center of a petri dish. The petri dish was inserted into an incubator at the designated CO₂ and temperature values for over twenty minutes. Dishes were subsequently washed with purified water to remove residue and allowed to air dry before storing at 4° C. if not being used immediately for experimentation. Cells were seeded directly onto the CTA-coated zone in the center of the petri dish. Cells were given a 20 minute incubation period to facilitate immobilization, before culture residue was washed off with PBS and dishes topped-up with appropriate media. Seeding cell culture containing FBS required pellet-resuspension into fresh, FBS-free media, to prevent CTA coating from preferentially binding to FBS. FBS was later supplied in the topped-up media once cells were allowed to immobilize in the incubation conditions.

Nanoindenter Installation

The nanoindenter instrument was attached perpendicular to a mounting post positioned vertically on an anti-vibration table. This table was connected at an inlet to an air compressor pump, supplying a designated compressed air pressure value via air hoses to isolation mounts positioned at the four corners of the table frame. The initial air supply pumped to the isolation mounts was controlled by an air pressure regulator. The height of the anti-vibration table's breadboard on top of the active isolation support frame was adjusted once the pump outlet supply provided sufficient air to raise the breadboard above the frame surface. The nanoindenter instrument was firmly positioned by bolting down the post it was connected to, to reduce any potential vibrational input. The head of the nanoindenter was then brought in-line with the objective lens of an inverted microscope to allow a clearer view of the region underneath the microscope to be probed. The nanoindenter was connected by cables to a interferometer and controller box, positioned alongside a dedicated laptop connected to these hardware.

Nanoindenter Experimentation

Experimental parameters were set on the nanoindenter software prior to experimentation. A specific nanoindenter probe was selected and mounted to the system based on the appropriateness of its dimensions for indenting a small single-cell. A calibration procedure was performed by positioning the nanoindenter probe in Cell Line A media. The surface of the petri dish was determined during calibration. A FS procedure was created to find the sample surface to determine the distance of the resting nanoindenter from the nearby targeted sample. Indentation depths between sample experiments were dynamic and dependent on the recorded FS distance each separate day. Standard indentations had a displacement around this FS distance 0.5 seconds into the experiment, moving downwards for 2 seconds before holding contact on the sample surface for 1 second, raising back upwards for another 2 seconds and then remaining stationary at the initial position prior to experimental run. The extended indentation procedure only differed by a 5-second holding time in contact with the sample surface. The depth management procedure acknowledged the average cell diameter recorded by the bioanalyzer for Cell Line A, indenting just below the maximum indentation depth of 16% of the probe tip-radius and 10% of sample thickness. MIOF were set at 1F, 2F, 4F and 10F Hz, with relaxation periods of 2 seconds between each increased frequency. The amplitude of the oscillation frequency was set at the system's default amplitude. The SOI were distanced 2 μm apart in 6 programmed directions over each targeted cell. A stored vial of Cell Line A was thawed into a shake flask at a designated rpm for a 72-hour period. Samples over three days were obtained after 26 hours, 46 hours and 72 hours and subsequently seeded onto prepared CTA-coated dishes for nanoindentation.

While in the foregoing specification these inventions have been described in relation to certain embodiments thereof, and many details have been put forth for the purpose of illustration, it will be apparent to those skilled in the art that the inventions are susceptible to additional embodiments and that certain of the details described herein can be varied considerably without departing from the basic principles of the inventions.

1. A method of quantifying the impact of shear stress on cells, wherein the method comprises the steps of
(a) exposing immobilized cells to forces that cause shear stress; and
(b) nanoindenting the cells from step (a) to determine their mechanical properties at different stress levels.

2. The method according to claim 1, wherein the cells are mammalian cells.

3. The method according to claim 2, wherein the mammalian cells are Chinese Hamster Ovary (CHO) cells, Baby hamster kidney (BHK) cells, Human embryonic kidney 293 (HEK293) cells, HeLa cells, per.c6 cells, nonsecreting murine myeloma (NSo) cells, or Sp2/0 murine myeloma cells.

4. The method according to claim 1, wherein the cells are suspension cells.

5. The method according to claim 1, wherein the cells are immobilized using a cell and tissue adhesive.

6. (canceled)

7. The method according to claim 5, wherein the cell and tissue adhesive is Cell-Tak.

8. The method according to claim 1, wherein the forces that cause shear stress to cells are generated by bioreactor agitation or shake flask agitation.

9. (canceled)

10. The method according to claim 1, wherein nanoindenting the cells is performed by a nanoindenter.

11. The method according to claim 10, wherein the nanoindenter comprises an optical probe.

12. The method according to claim 11, wherein the optical probe comprises a cantilever.

13. The method according to claim 11, wherein the probe is mechanically lowered from a pre-calibrated distance toward the surface of the cells.

14. The method according to claim 13, wherein the probe is lowered for a period of two seconds.

15. The method according to claim 12, wherein upon contact with the cantilever, the cell exerts a force upon the cantilever causing the cantilever to bend.

16. The method according to claim 15, wherein the probe is mechanically raised for a period of two seconds.

17. The method according to claim 15, wherein the cantilever is in contact with the cell surface for at least one second.

18. (canceled)

19. The method according to claim 18, wherein upon contact with the cell, the cantilever generates multiple increasing oscillation frequencies of 1F Hz, 2F Hz, 4F Hz, and 10F Hz.

20. The method according to claim 19, wherein no oscillation frequency is generated for a period of two seconds between the generation of each increasing oscillation frequency.

21. The method according to claim 10, wherein the nanoindenter subjects the cells to six rounds of nanoindentation.

22. The method according to claim 21, wherein each subsequent nanoindentation is placed 2 μm from the preceding nanoindentation.

23. The method according to claim 1, wherein the mechanical properties of the cells are determined after nanoindentation.

24. The method according to claim 1, wherein the mechanical properties of the cells comprise cell stiffness.

25. The method according to claim 24, wherein cell stiffness is determined by calculating Young's modulus (YM) and Effective Young's modulus (EYM).

26. The method according to claim 25, wherein the YM and EYM of cells after 26 hours and 46 hours of shear stress is less than about 50×Pa.

27. (canceled)

28. The method according to claim 25, wherein the YM and EYM of cells after 72 hours of shear stress is greater than about 500×Pa.

29. The method according to claim 24, wherein cell stiffness is determined by calculating storage modulus (E′) or loss modulus (E″).

30. (canceled)

31. The method according to claim 29, wherein the E′ value is higher than the E″ value at frequencies of 1F, 2F, and 10F Hz after at least two days of agitation, indicating elasticity of the cells.

32. The method according to claim 29, wherein the E″ value is higher than the E′ value at a frequency of 4F Hz after at least two days of agitation, indicating viscosity of the cells.

33. A process of bioproduction optimization, the process comprising:
(a) inflicting shear stress on cells;
(b) quantifying the impact of shear stress on cells according to the method of claim 1; and
(c) using the data obtained from step (b) to adjust the levels of shear force applied during bioproduction.

34. The process of claim 33, wherein optimization results in an increase in:
(a) product titer and yield;
(b) cell viability; and/or
(c) product quality,
wherein product quality is determined by glycosylation efficiency.

35.-37. (canceled)

38. A method of developing cell lines that are resistant to shear stress, the method comprising
(a) inflicting shear stress on said cells with increasing levels of shear force;
(b) quantifying the impact of shear stress on cells according to the method of claim 1; and
(c) selecting resistant cells from step (b) for further use in bioproduction.
